(19) United States
(12) Patent Application Publication
(10) Pub. No.: US 2022/0296250 A1
(43) Pub. Date: Sep. 22, 2022

(54) Insufflation Retention Device with Balloon and other Features

(71) Applicant: BPENDO, LLC, Norman, OK (US)

(72) Inventors: John Hayes, Morrisville, NC (US); Robert Henson, Morrisville, NC (US); Amanda Schaffers, Morrisville, NC (US); Robert M Holbrook, Norman, OK (US)

(21) Appl. No.: 17/701,660

(22) Filed: Mar. 22, 2022

Related U.S. Application Data
(60) Provisional application No. 63/164,519, filed on Mar. 22, 2021.

(51) Int. Cl. A61B 17/12

(52) U.S. Cl. CPC A61B 17/12136; A61B 17/1204; A61B 17/12099; A61B 2017/1205

(57) ABSTRACT

A probe may be inserted into a body cavity to perform diagnostic intervention(s), therapeutic intervention(s), or both. The probe may be inserted through a body aperture that is naturally occurring or man-made, intentionally or by accident. The body aperture may form a seal encircling the probe so that insufflation retention material may be effectively retained in the body cavity so that an operator can perform the intervention(s). However, there may be leakage of the insufflation material. The insufflation retention device is configured to form an effective seal contactingly adjacent the body aperture and to provide a passageway for the introduction of the probe into the body cavity, such that a diagnostic intervention or therapeutic intervention or both may be performed.

### CROSS-REFERENCE TO RELATED APPLICATIONS

The present application claims priority to U.S. Prov. Pat. App. No. 63/164,519 entitled Insufflation Retention Device with Balloon and other Features, which was filed Mar. 22, 2021, and was copending with the present application. The U.S. Prov. Pat. App. No. 63/164,519 is hereby incorporated in its entirety into the present application.

### SUMMARY

In accordance with various embodiments, a probe may be inserted into a body cavity to perform diagnostic intervention(s), therapeutic intervention(s), or both. The probe may be inserted through a body aperture that is naturally occurring or man-made, intentionally or by accident. The body aperture may form a seal encircling the probe so that insufflation retention material may be effectively retained in the body cavity so that an operator can perform the intervention(s), in which case a body probe seal is considered competent. However, there may be leakage of the insufflation material, in which case the body probe seal is considered incompetent. The insufflation retention device is configured to form an effective seal contactingly adjacent the body aperture and to provide a passageway for the introduction of the probe into the body cavity to create a competent seal between the body aperture and the insufflation retention device and another competent seal between the probe and the insufflation retention device.

### BRIEF DESCRIPTION OF THE DRAWINGS

FIGS. 1A-1E show side views of an apparatus that is an insufflation retention device in accordance with various embodiments of the present disclosure.

FIGS. 2A-2E show end views of the insufflation retention device in accordance with various embodiments of the present disclosure.

FIG. 3 shows an isometric view of an exploded embodiment of the insufflation retention device in accordance with various embodiments of the present disclosure.

FIG. 4 shows a side view of the handle of the insufflation retention device in accordance with various embodiments of the present disclosure.

FIG. 5 shows a close-up of the expansion material port and the expansion material open channel.

FIG. 6 shows an end view of the external buttress of the insufflation retention device in accordance with various embodiments of the present disclosure.

FIG. 7 shows a cross-section of FIG. 6 of the handle of the insufflation retention device in accordance with various embodiments.

FIG. 8 shows a close-up of a cross-section of FIG. 7 of the expansion material port and the expansion material open channel in accordance with various embodiments.

FIG. 9 shows a cross-section of the handle of the insufflation retention device of in accordance with various embodiments.

FIG. 10 shows a close-up of the knife edge channel of FIG. 9.

FIG. 11 shows an end view of the external buttress with the balloon of the internal buttress 106 extending into the passageway of the insufflation retention device in accordance with various embodiments of the present disclosure.

FIG. 12 shows a partial cross-section of the handle with the internal buttress and the external buttress of the insufflation retention device in accordance with various embodiments of the present disclosure.

FIG. 13 shows a close-up of FIG. 12 in which the expansion material port is in fluid communication with the expansion material open channel enclosed by the internal buttress.

FIG. 14 shows a side view of the insufflation retention device with the external buttress 102 and the internal buttress in accordance with various embodiments of the present disclosure.

FIG. 15 shows a cross-section of FIG. 14 looking towards the external buttress 102.

FIG. 16 shows a close-up of FIG. 15 of the expansion material port.

FIG. 17 shows a cross-section of FIG. 14 towards the internal buttress.

FIG. 18 shows a close-up of FIG. 18 of the expansion open channel.

FIG. 19 shows a custom isometric view of the insufflation retention device in accordance with various embodiments of the present disclosure.

FIGS. 20A-D shows side views of the insufflation retention device in accordance with various embodiments of the present disclosure.

### DETAILED DESCRIPTION

There are technologies that allow operators to introduce a probe, e.g., a medical scope, into a body cavity for diagnostic intervention or therapeutic intervention or both. When the probe is introduced, the body cavity may need to be expanded for the operator to perform the intervention(s). Using an insufflation technique, the operator may introduce an insufflation material to expand the body cavity, so the operator may have more room to work and better visibility in the body cavity to perform the intervention(s). E.g., see Technology Status Evaluation Report: Methods of luminal distension for colonoscopy, Gastrointestinal Endoscopy, Volume 77, No. 4, 2013, pages 519-525, which is incorporated by reference in its entirety. The insufflation material may be air, carbon dioxide, water, or other suitable materials.

The operator may start with the probe outside a body, and the operator may advance the probe through tissue of the body to introduce the probe into a cavity of the body, i.e., the body cavity. The probe may be advanced through the tissue via an aperture of the body, i.e., the body aperture, that is a naturally occurring orifice, e.g., an anus, or a wound, e.g., a surgical incision or a traumatic injury. The body aperture may have elasticity that allows the body aperture to recover its size and shape after any deformation from the probe being advanced through the body aperture into the body cavity to effectively seal the outside of the body from the body cavity. Thereafter, the insufflation material introduced into the body cavity may be retained in the body cavity to help promote expansion of the body cavity when the outside of the body is effectively sealed from the body cavity to permit the operator to perform the intervention(s).

However, the insufflation material may not be effectively retained in the body cavity in some instances. For example, the body aperture or nearby structures may have a congenital malformation or may have suffered structural injury such as from scar tissue formation after abscess formation, surgical trauma, giving birth related injury, etc. that inhibits the body aperture from forming an effective seal with the probe.

If the insufflation material is not effectively retained, then the operator will not have time and room to work or visibility to operate in the body cavity. For example, the probe, such as an endoscope, may be introduced into the body cavity, such as a rectum and a large intestine, through the body aperture, such as the anus, and the elasticity of the body aperture may not effectively form a seal contactingly adjacent the probe to promote retention of the insufflation material in the body cavity. As will be described in further detail, this disclosure describes an insufflation retention device that promotes retention of the insufflation material in the body cavity with or without any abnormality of the body aperture.

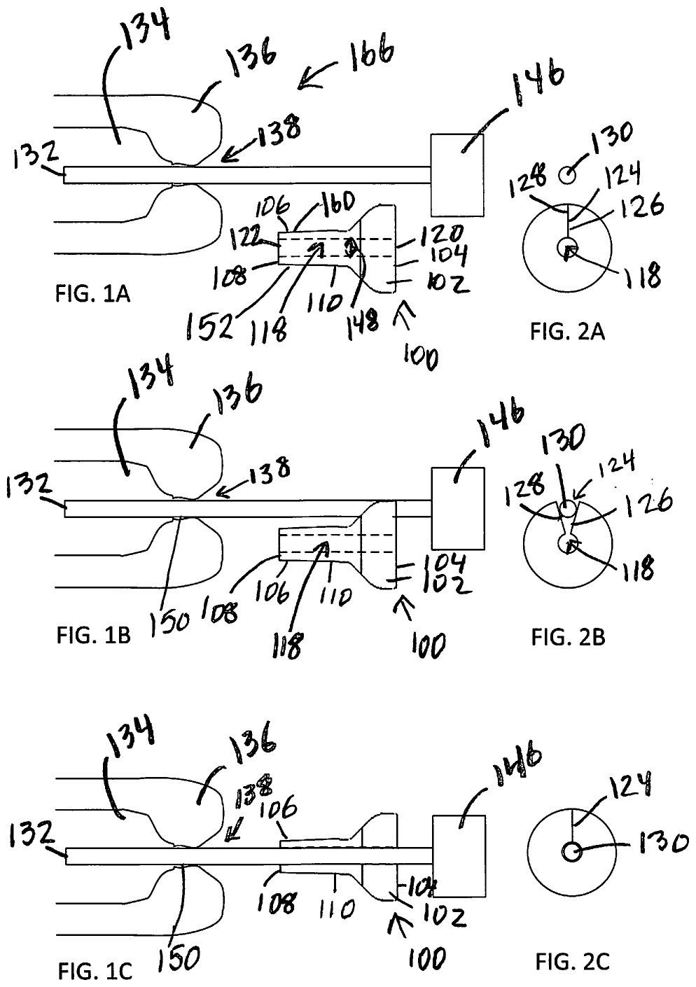

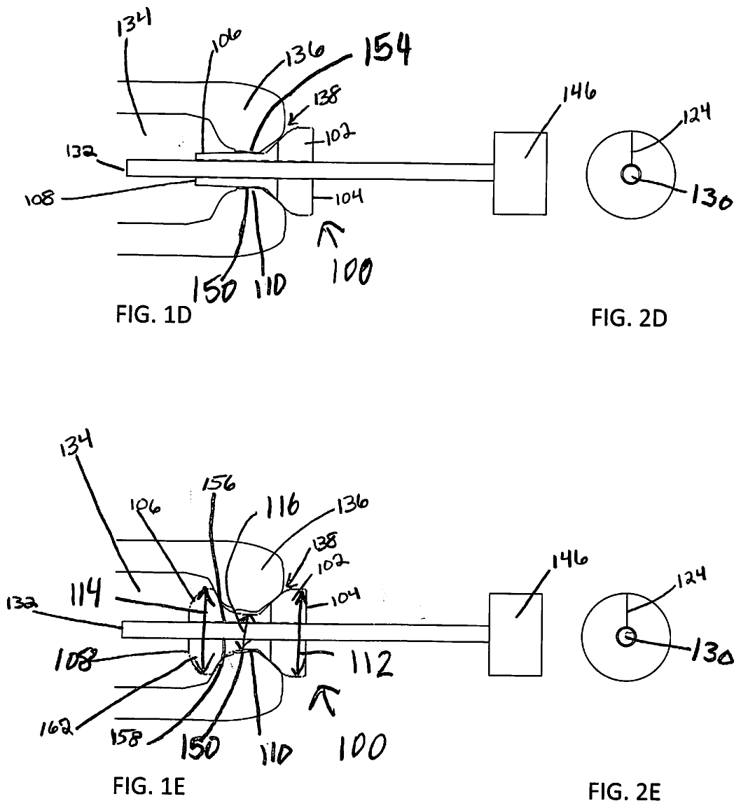

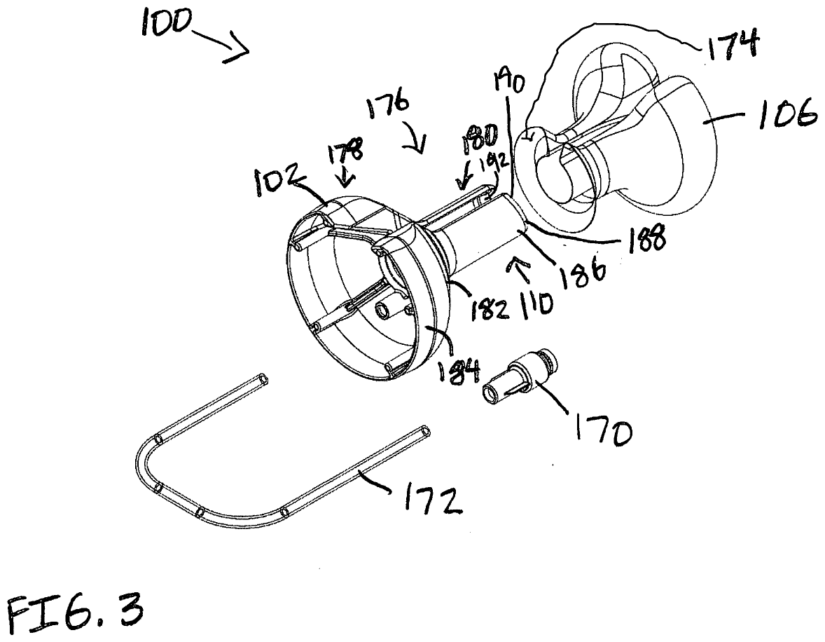

FIGS. 1A-1E show side views of an apparatus that is an insufflation retention device (also known herein as IRD 100) in accordance with various embodiments of the present disclosure. FIGS. 2A-2E show end views of the IRD 100.

Optionally, the IRD 100 may include an external buttress 102 at an outside end 104. Optionally, the IRD 100 may include an internal buttress 106 at an inside end 108. The inside end 108 may be opposite the outside end 104. The external buttress 102 may be coupled to the internal buttress 106.

Optionally, there may be a midportion 110 situated between the external buttress 102 and the internal buttress 106. The midportion 110 may be configured to couple the external buttress 102 to the internal buttress 106.

An external buttress width 112 of the external buttress 102 may be substantially the same as, substantially greater than, or substantially less than an internal buttress width 114 of the internal buttress 106. A midportion width 116 of the midportion 110 may be substantially less the external buttress width 112 and the internal buttress width 114. The external buttress width 112, the internal buttress width 114, and the midportion width 116 may be substantially parallel to each other.

Optionally, the IRD 100 may include a passageway 118. The passageway 118 may extend from an external buttress aperture 120 in the external buttress 102 to an internal buttress aperture 122 in the internal buttress 106. The passageway 118 may extend from the external buttress aperture 120 through the midportion 110 to the internal buttress aperture 122. The passageway 118 may be substantially circular, and the passageway 118 may be substantially symmetrically disposed with the IRD 100.

Optionally, the IRD 100 may include a seam 124. The seam 124 may extend from the external buttress 102 to the internal buttress 106. The seam 124 may extend from the external buttress aperture 120 to the internal buttress aperture 122. The seam 124 may extend from the external buttress 102 through the midportion 110 to the internal buttress 106. The seam 124 may extend from the external buttress aperture 120 through the midportion 110 to the internal buttress aperture 122. The seam 124 is space between a first adjacent edge 126 of the IRD 100 and a second adjacent edge 128 of the IRD 100.

A probe 130 may have a tip 132 configured for insertion into a body cavity 134 of a body 136 through a body aperture 138 of the body 136. An operator of the probe 130 may be able to slide the tip 132 into the passageway 118 of the IRD 100 when the tip 132 is not in the body cavity 134. However, when the tip 132 is in the body cavity 134, the operator of the probe 130 will not be able to slide the tip 132 into the IRD 100. In addition, the probe 130 may have a closed end 146 that is opposite the tip 132. The closed end 146 may include electronics, controls, connections to other devices, etc. The operator may not be able to slide the closed end 146 into the passageway 118, because the closed end 146 is bigger than a diameter 148 of the passageway 118. The body aperture 138 may have an abnormality 150, such that insufflation material may not be effectively retained in the body cavity.

Rather than remove the probe 130 from the body cavity 134 to the position the probe 130 in the passageway 118, the operator may use the seam 124 to position the probe 130 in the passageway 118. The seam 124 may be biased towards a closed state. When the seam 124 is in the closed state, the first adjacent edge 126 and the second adjacent edge 128 may be contactingly adjacent. When the seam 124 is in the closed state, the first adjacent edge 126 and the second adjacent edge 128 may be closely adjacent, but not contactingly adjacent. When the seam 124 is in the closed state, the operator will not be able to slide the probe 130 past the first adjacent edge 126 and the second adjacent edge 128 into the passageway 118. On the other hand, the operator may be able to move the seam 124 from the closed state to an open state. When the seam 124 is in the open state, the operator will be able to slide the probe 130 past the first adjacent edge 126 and the second adjacent edge 128 into the passageway 118. Then, the probe 130 can be positioned in the passageway 118 without the operator needing to slide the tip 132 into the passageway 118 or slide the closed end 146 into the passageway 118. In other words, the IRD 100 may be placed around the probe 130 while the tip 132 remains in the body cavity 134 so the tip 132 cannot slide into the passageway 118 and the closed end 146 of the probe 130 cannot slide into the passageway 118, which will be convenient for the operator.

When the IRD 100 is positioned through the body aperture 138 into the body cavity 134, the seam 124 may be helped to stay in the closed state through the pressure from the body 136 on the IRD 100. Further, an external surface 152 of the IRD 100 may form an IRD body seal 154 and an internal surface 156 of the IRD 100 may form an IRD probe seal 158. The IRD body seal 154 and the IRD probe seal 158 may be configured to help retain insufflation material to expand the body cavity 134, so the operator may have more room to work and better visibility in the body cavity 134 to perform the intervention(s).

The internal buttress 106 may be configured to have an unexpanded state 160 so that the operator may introduce the IRD 100 through the body aperture 138 into the body cavity 134. The unexpanded state 160 of the internal buttress 106 may be smaller than an expanded state 162 of the internal buttress 106. The unexpanded state 160 of the internal buttress 106 may be configured to facilitate ingress of the IRD 100 from an exterior 166 of the body 136. In other words, the unexpanded state 160 of the internal buttress 106 may be a contracted state of the internal buttress 106 that may be configured for ingress, or entry, through the body aperture 138 of the body 136 into the body cavity 134 of the body 136.

The expanded state 162 of the internal buttress 106 may configured to prevent egress of the IRD 100 from the body cavity 134. If the IRD 100 moved towards the exterior 166 of the body 136, then the expanded state 162 of the internal buttress 106 would contactingly engage the body cavity 134 or the body aperture 138 or both to prevent egress of the IRD 100 from the body cavity 134. In other words, the expanded state 162 of the internal buttress 106 that may be configured to inhibit egress, or removal, of the internal buttress 106 from the body cavity 134 through the body aperture 138.

The internal buttress 106 in the unexpanded state 160 or contracted state may be increased in size to the expanded state 164 through introduction of an expansion material into an internal cavity of the internal buttress 106 supplied by a source discussed later in further detail. The expansion material may be broadly considered to be a fluid. Examples of the expansion material may be a liquid e.g., water, and/or a gas e.g., oxygen, air, compressed air, carbon dioxide, by way of example and not limitation.

FIG. 3 shows an isometric view of an exploded embodiment of the IRD 100 in accordance with various embodiments of the present disclosure. Optionally, the IRD 100 may include a valve 170, such as but not limited to a Luer-Lok brand activated valve (Luer-Lok is a brand name owned by BECTON, DICKINSON AND COMPANY). The valve 170 may be attached to any suitable source of expansion material, such as but not limited to a syringe, gas line, water line, etc. The valve 170 may be configured to be closed (to block flow) or open (to permit flow) or both when the IRD 100 is connected or not connected to the source. Optionally, the IRD 100 may include a tubing 172 that may be configured to convey the expansion material from the any suitable source to the internal buttress 106.

The IRD 100 may be manufactured using any suitable technique. For example, the IRD 100 could be manufactured with a blow molding technique of the external buttress 102, the internal buttress 106, and the midportion 110 as one piece. However, it may be easier to manufacture the IRD 100 using a two-part construction technique for the external buttress 102, the internal buttress 106, and the midportion 110.

The internal buttress 106 may be manufactured using a mandrel in a dip molding process, by way of example and not limitation. The internal buttress 106 may be considered a balloon with an open end 174. The internal buttress 106 may be manufactured in the open state.

The external buttress 102 may be manufactured using an injection molding process, by way of example and not limitation. The external buttress 102 may be manufactured as part of what may be characterized as a handle 176, given that the handle 176 may be configured to be the most rigid portion of the IRD 100. Optionally, the handle 176 may include a bell portion 178 that may be configured to extend to a shaft portion 180. Optionally, the bell portion 178 may be further divided into a skirt portion 182 and a bow portion 184. The shaft portion 180 may form part of the midportion 110, and the shaft portion 180 may extend into the internal buttress 106. Optionally, the handle 176 may include an introducer 186 that may be configured to decrease puncture force of the IRD 100 insertion into the body cavity 134 through the body aperture 138 (see FIGS. 1-2). A leading edge 190 of the introducer 186 may be configured to be rounded from an exterior surface 188 to an interior surface 192 to reduce incidents of mechanical stress, tearing, etc. on the internal buttress 106 by the handle 176.

When the two-part construction is used, the external buttress 102 and the midportion 110 may be configured to be manufactured in the closed state. As will be discussed in further detail later, the external buttress 102 and the midportion 110 may be configured to be cut, after which there will be a seam 124 in the external buttress 102 and the midportion 110. Further, the internal buttress 106 may be configured to be manufactured in the open state, or at least a partially open state. Then, the handle 176 may be configured to be positioned in the partially open state to substantially match the internal buttress 106 in the partially open state, so that the handle 176 may be slid into the open end 174 of the internal buttress 106. Then, the handle 176 may be fastened to the internal buttress 106 using any suitable technique, such as welding, soldering, adhesive bonding, solvent bonding, etc., by way of example and not limitation. After which, the handle 176 may be released from the partially open state. Then, the handle 176 may be configured to assume the closed state due to the bias to the closed state in which the handle 176 was manufactured. Further details are discussed later.

The IRD 100 may be made of any suitable biologically compatible material, or biocompatible material, including, by way of example and not limitation, polymers, such as thermoplastic polyurethane (TPU), thermoplastic elastomer (TPE), polysiloxane or silicone, and low-density polyethylene LDPE, and other natural or synthetic rubbers. The various parts of the IRD 100 may be made of the same or different materials. Manufacturing the handle 176 and the internal buttress 106 of the same material may be useful to maintain similar mechanical properties between the handle 176 and the internal buttress 106.

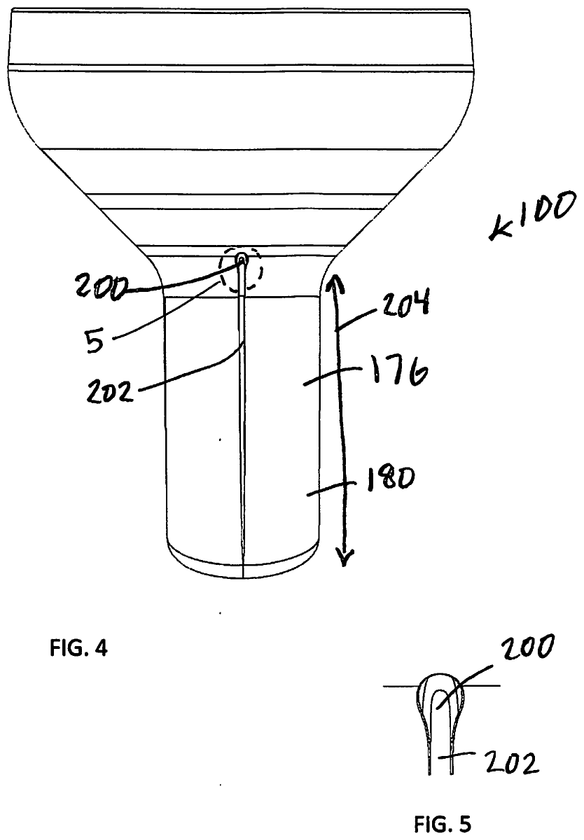

FIG. 4 shows a side view of the handle 176 of the IRD 100 in accordance with various embodiments of the present disclosure. Optionally, the handle 176 may include an expansion material port 200 and an expansion material open channel 202 that extends from the expansion material port 200. The expansion material port 200 may be configured for insertion of the expansion material into the IRD 100 and may be configured for evacuation of the expansion material from the IRD 100. The may take any suitable configuration. As shown, the expansion material open channel 202 may be configured to extend from the expansion material port 200 for most of a length 204 along the shaft portion 180. While the expansion material open channel 202 may be configured to be wider towards the expansion material port 200 and narrower as the expansion material open channel 202 extends away from the expansion material port 200, other configurations are possible. In addition, dimensions and configuration of the expansion material open channel 202 may be configured to optimize insertion and evacuation of the expansion material through the expansion material port 200. Dimensions and configuration of the expansion material open channel 202 may account for the “film” characteristics of the internal buttress 106 (see FIG. 3). If the expansion material open channel 202 is too wide or too shallow or both, then the expansion material open channel 202 may not be configured to effectively insert or evacuate the expansion material, because the internal buttress 106 may “cling” to the handle 176 in the expansion material open channel 202.

FIG. 5 shows a close-up of the expansion material port 200 and the expansion material open channel 202. As would be understood by one skilled in the art, flow of the expansion material in the expansion material port 200 would have a type of flow characteristic of pipe flow when the expansion material is inserted or evacuated. As would be understood by one skilled in the art, flow of the expansion material in the expansion material open channel 202 would have a type of the flow characteristic of channel flow with a free surface when the expansion material is inserted or evacuated.

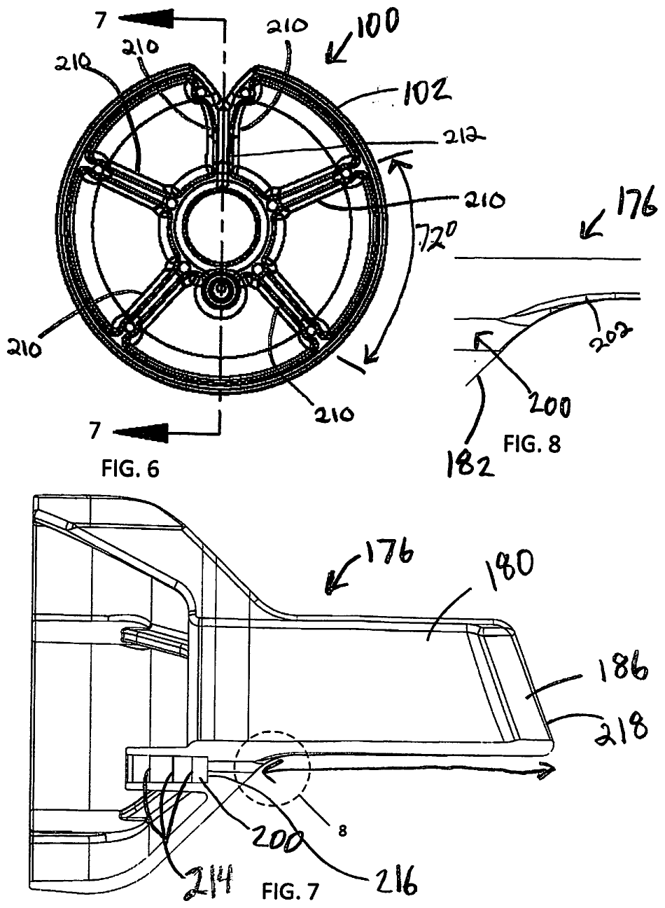

FIG. 6 shows an end view of the external buttress 102 of the IRD 100 in accordance with various embodiments of the present disclosure. Optionally, the IRD 100 may include one or more of a strut 210, wherein the strut 210 is configured to resist pressure in the direction of its length. The one or more of the strut 210 may enable the bell portion 178 to be thinner than it might otherwise be able to be and still provide a configuration of the external buttress for proper operation of the IRD 100. For example, the external buttress 102 may be configured to be of substantially the same shape before, during, and after use of the IRD 100. In other words, the external buttress 102 may be configured to have only a state that could be considered the expanded state. Of course, the external buttress 102 could be configured to have the unexpanded state and the expanded state through similar, expansion material port, expansion material open channel, balloon, etc. that are used for the internal buttress 106 (FIG. 3). In which case, the external buttress 102 may be configured to have a fastener to assist the external buttress 102 and to achieving the closed state. The fastener may include a strap, hook and loop, etc. by way of example and not limitation.

The IRD 100 may of course have one or more of the expansion material port 200; however, one of the expansion material port 200 may be all that is necessary. As shown in FIG. 6, the expansion material port 200 may be configured to be substantially opposite a cut line 212 of the external buttress 102. In positioning the expansion material port 200 substantially opposite the cut line 212, the operator may find it easier to splay open the IRD 100 from the closed state to the open state. If the expansion material port 200 is configured to be closer to the cut line 212, mechanical properties of the expansion material port 200 may make it harder to splay open the IRD 100.

While the IRD 100 may include one or more of the strut 210, five of the strut 210 may be configured to be approximately 72° apart, such that one of the strut 210 does not coincide with a position of the expansion material port 200. Along the cut line 212, one of the strut 210 may be configured to be approximately twice as wide as others of the strut 210, such that when the double wide of the strut 210 is cut along the cut line 212, the IRD 100 may be configured to have each of the strut 210 of any suitable width. And the IRD 100 would go from having five of the strut 210 in the uncut condition and having six of the strut 210 in the cut condition. Of course, each of the strut 210 may be varied in dimension and configuration as needed and each strut may be different from the other.

FIG. 7 shows a cross-section of FIG. 6 of the handle 176 of the IRD 100 in accordance with various embodiments. Optionally, the expansion material port 200 may be configured to have one or more of a barb 214 that is configured to engage the tubing 172 (see FIG. 3). The presence of one or more of the barb 214 may eliminate a component connector and potentially a bonding step, such as welding, soldering, adhesive, etc. by way of example and not limitation, of the tubing 172 to the expansion material port 200 in the manufacture of the IRD 100. Optionally, the expansion material port 200 may narrow and have an end stop 216, as shown, configured to keep the tubing 172 from sliding all the way through the expansion material port 200.

Optionally, the handle 176 may include the introducer 186, as shown. The introducer 186 may have a bevel 218, which is a surface that may be any angle that is not substantially a right angle to the length 204 along the shaft portion 180.

FIG. 8 shows a close-up of a cross-section of FIG. 7 of the handle 176 in accordance with various embodiments. The expansion material open channel 202 may be configured to extend from the expansion material port 200 along a swept path that cuts into the skirt portion 182 of the handle 176 and then along at least a portion if not substantially all of the shaft portion 180 of the handle 176. The expansion material open channel 202 is configured to “tunnel” under the internal buttress 106 to collect as much air as possible for faster evacuation of the balloon.

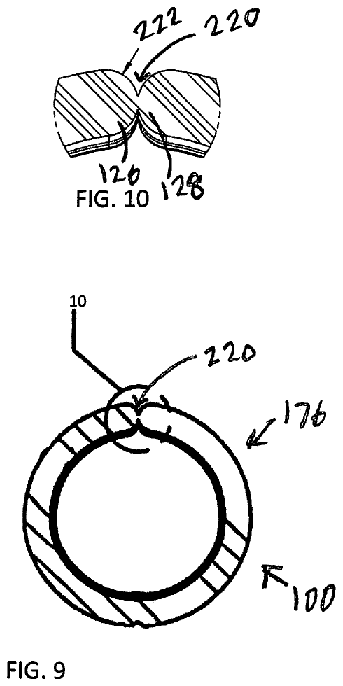

FIG. 9 shows a cross-section of the handle 176 of the IRD 100 in accordance with various embodiments. Optionally, the handle 176 may be molded with a knife edge channel 220 with a rounded lead-in 222 on the first adjacent edge 126 and the rounded lead-in 222 on the second adjacent edge 128. FIG. 10 shows a close-up of the knife edge channel 220 of FIG. 9. The knife edge channel 220 may be configured to closely match any contour of the internal buttress 106, such as might be manufactured through the dip molding process. The internal buttress 106 may be manufactured to perform as a balloon, which may not have hard edges due to manufacturing constraints. Further, manufacturing limitations exist with injection molding of the handle 176 with regards to thickness of the wall between an external surface and an internal surface. Therefore, the handle 176 may not be able to be molded in a split state with the seam 124 (see FIG. 3). The knife edge channel 220 may solve this manufacture limitation by allowing the handle 176 to be manufactured with the rounded lead-in 222 on the first adjacent edge 126 and the rounded lead-in 222 on the second adjacent edge 128. The rounded lead-in 222 to the seam 124 that may be cut as a secondary process. In other words, the handle 176 may be manufactured with the knife edge channel 220 and the seam 124 is absent and in the cutting process along the knife edge channel 220, the seam 124 becomes present or is created.

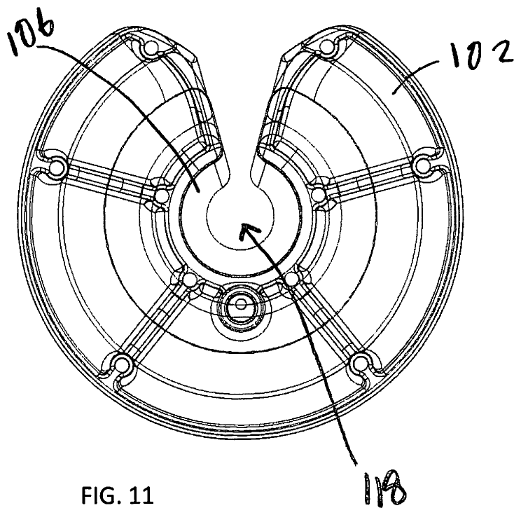

FIG. 11 shows an end view of the external buttress 102 with the balloon of the internal buttress 106 extending into the passageway 118 of the IRD 100 in accordance with various embodiments of the present disclosure.

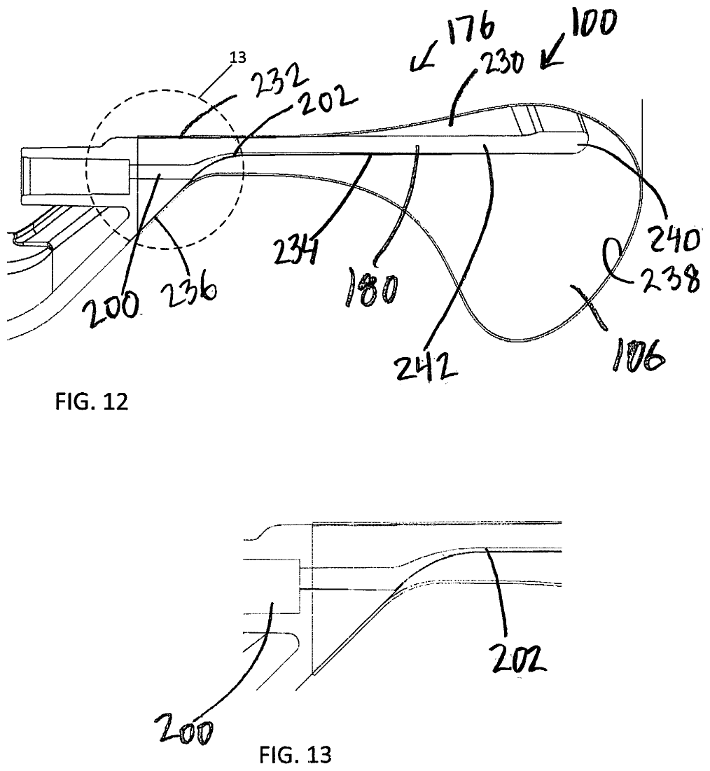

FIG. 12 shows a partial cross-section of the handle 176 of the IRD 100 in accordance with various embodiments of the present disclosure. The internal buttress 106 may be configured to be fastened to an internal handle surface 230 of the handle 176 at an internal fastening location 232. The internal buttress 106 may be configured to fasten to an external handle surface 234 of the handle 176 at an external fastening location 236. The expansion material port 200 may extend to the expansion material open channel 202. The expansion material open channel 202 may be configured to be inside the internal buttress 106. Effectively, the expansion material open channel 202 may be inside a balloon. Expansion material may be configured to be contactingly adjacent an internal surface 238 of the internal buttress 106, the internal handle surface 230, and the external handle surface 234.

A leading edge 240 of the shaft portion 180 of the handle 176 may be configured to be enclosed by the internal buttress 106. The leading edge 240 may be configured to be rounded, as shown, to reduce the likeliness of the leading edge 240 puncturing the internal buttress 106 when the IRD 100 is inserted into the body aperture 138 (see FIGS. 1A-1E). A side edge 242 of the shaft portion 180 of the handle 176 may be configured to be enclosed by the internal buttress 106. The side edge 242 may be the first adjacent edge 126 (see FIG. 10) or the second adjacent edge 128 (see FIG. 10) or both.

FIG. 13 shows a close-up of FIG. 12 in which the expansion material port 200 is in fluid communication with the expansion material open channel 202 enclosed by the internal buttress 106.

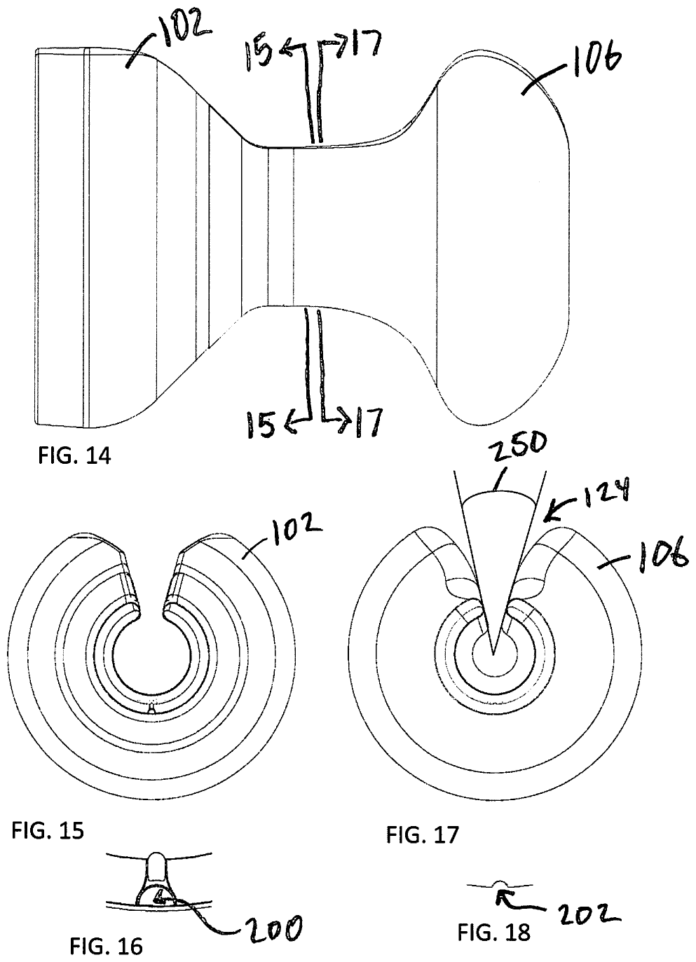

FIG. 14 shows a side view of the IRD 100 with the external buttress 102 and the internal buttress 106 in accordance with various embodiments of the present disclosure. The external buttress 102 is in the expanded state. The internal buttress 106 is in the expanded state. FIG. 15 shows a cross-section of FIG. 14 looking towards the external buttress 102. FIG. 16 shows a close-up of FIG. 15 of the expansion material port 200. FIG. 17 shows a cross-section of FIG. 14 towards the internal buttress 106. An opening angle 250 of the seam 124 may be of any suitable angle. As a reminder, the handle 176 (see FIG. 4) may be injection molded in the closed state and the internal buttress 106 may be dip molded in the open state for demolding or removal from the mold. The opening angle 250 may be manufactured in the partially open state with an angle of approximately 45° to approximately 20°. A suitable angle of opening angle 250 may be approximately 30°. In the manufacturing process there may be a trade-off between challenges in producing the opening angle 250. If the opening angle 250 is too small there may be challenges in inserting the handle 176 into the internal buttress 106. If the opening angle 250 is too large there may be challenges in opening a handle 176 enough to insert the handle 176 into the internal buttress 106. FIG. 18 shows a close-up of FIG. 17 of the expansion material open channel 202.

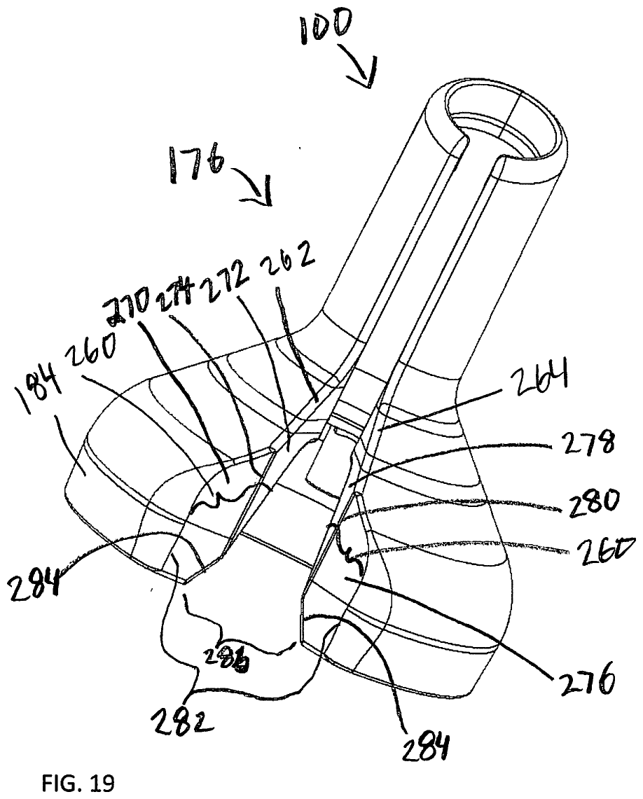

FIG. 19 shows a custom isometric view of the IRD 100 in accordance with various embodiments of the present disclosure. Optionally, the handle 176 may have a side lead-in 260 on a first side 262, which may be characterized as a first side lead-in 260′, of the bow portion 184, and the handle 176 and the 260 on a second side 264, which may be characterized as a second side lead-in 260″, of the bow portion 184. Optionally, the side lead-in 260 on the first side 262 may have a first surface 270 and a second surface 272. There may or may not be a well-defined transition edge 274 between the first surface 270 and the second surface 272. The well-defined transition edge 274 is seen in FIG. 19. Optionally, the side lead-in 260 of the second side 264 may have a first surface 276 and a second surface 278. There may or may not be a well-defined transition edge 280 between the first surface 276 and the second surface 278. The well-defined transition edge 280 is seen in FIG. 19. Each of the first surface 270, the second surface 272, the first surface 276, and the second surface 278 may be substantially planar, convex, or concave. The second surface 272 of the first side 262 and the second surface 278 of the second side 264 may be formed in the secondary cutting process discussed hereinabove in relation to FIGS. 9 and 10. Collectively, the side lead-in 260 of the first side 262 and the side lead-in 260 of the second side 264 may be characterized as a probe side lead-in 282.

In addition, the handle 176 may have an end lead-in 284 on the first side 262, which may be characterized as a first end lead-in 284′, of the bow portion 184, and the handle 176 may have the end lead-in 284 on the second side 264, which may be characterized as a second end lead-in 284″, of the bow portion 184. The first end lead-in end 284′ and the second end lead-in 284″ may be substantially planar, convex, or concave. Collectively, the first end lead-in 284′ and the second end lead-in 284″ may be characterized as an end lead-in 286.

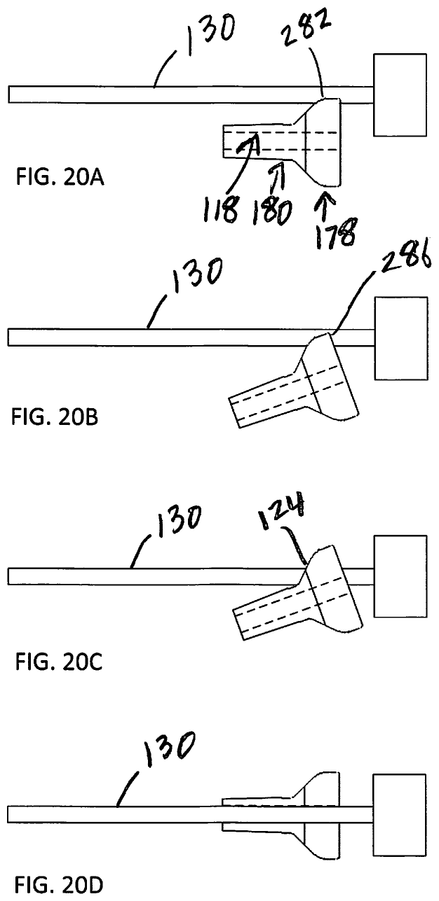

FIGS. 20A-D shows side views of the IRD 100 in accordance with various embodiments of the present disclosure. FIG. 20A shows the probe 130 engaged with the probe side lead-in 282. The IRD 100 may be configured to be in the closed state as the probe 130 is configured to nestle into the probe side lead-in 282. FIG. 20B shows an interesting operation of the end lead-in 286. As the operator moves the probe 130 towards the passageway 118, the operator may feel resistance in the transition from the closed state of the IRD 100 to the open state of the IRD 100. The shaft portion 180 and the bell portion 178 may be both biased to the closed state. However, the IRD 100 may be configured to have the probe 130 engage the bell portion 178 sooner than the shaft portion 180. In addition, as the IRD 100 may be configured to have probe 130 engage the bell portion 178 and there is a natural tendency of the probe 130 to pivot relative to the IRD 100 at the probe end lead-in 286. This pivot of the probe 130 relative to the IRD 100 may be configured to help splay open the seam 124, best seen in other FIGS (such as FIG. 2B). FIG. 20C shows that the IRD 100 may be configured to maintain pivot relative to the probe 130 at the probe end lead-in 286, as the probe 130 is pushed further towards the passageway 118 by the operator. The seam 124 may be further splayed open. FIG. 20D shows the probe 130 has completed the transition from outside the IRD 100 into the passageway 118 of the IRD 100 such that the probe 130 is approximately fully surrounded by the IRD 100. In other words, the IRD 100 has transitioned from the closed state, to the open state to allow entry of the probe 130 into the passageway 118, and back to the closed state with the probe 130 in the passageway 118.

One skilled in the art would understand that the probe may be an endoscope, by way of example and not limitation. The internal buttress may be configured to accommodate probes of various sizes: diameter, length, etc. A commercially available endoscope would have a light source configured to provide light in the lumen of a colon, such as body cavity 134, and an integrated air pump configured to provide air in the lumen of the colon for luminal expansion at colonoscopy. Furthermore, one skilled in the art would understand that the endoscope could be configured to use CO₂, water, or other suitable materials for insufflation of the lumen of the colon.

For colonoscopy, one skilled in the art would understand that bowel preparation quality may impact the success of colonoscopy. Many bowel preparation agents are available to accomplish adequate bowel cleanliness. E.g., Optimizing bowel preparation for colonoscopy: a guide to enhance quality of visualization, Ann Gastroenterol 2016; 29 (2): 137-146, which is incorporated by reference in its entirety.

Again, all of the limitations disclosed hereinabove are optional. Any of the features may be mixed and matched other than ones that obviously cannot be such as having an IRD 100 with the seam 124 that also does not have the seam 124.

By way of example and not limitation, the expansion material port 200 and the expansion material open channel 202 may be manufactured in the IRD 100 without the seam 124 that could be made by manufacture of the IRD 100 without the knife edge channel 220 or without the knife edge channel 220 that is then cut as the secondary process, such that the IRD 100 may be configured for the bell portion 178 to maintain a continuous surface and the shaft portion 180 to maintain a continuous surface around the passageway 118. Of course, then the internal buttress 106 would be manufactured without the seam 124, which could be easier to manufacture by the dip molding process without the seam 124. The internal buttress 106 would still be manufactured with the open end 174 so that the expansion material open channel 202 could be captured inside the internal buttress 106 when the handle 176 and the internal buttress 106 are combined in the two-part construction.

The IRD 100 may be manufactured by any suitable steps, such as but not limited to the steps presented.

It is to be understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. An insufflation retention device comprising:
an internal buttress configured to inhibit removal of the internal buttress from a body cavity through a body aperture of a body, wherein the internal buttress has an unexpanded state and an expanded state after introduction of an expansion material, and an internal surface and external surface;
an external buttress coupled to the internal buttress, the external buttress configured to inhibit entry of the external buttress into the body cavity through the body aperture;
a passageway extending through the internal buttress and the external buttress that is configured for passage of a probe in contacting engagement with the body cavity, wherein the passageway is configured to be open without the probe present in the passageway such that an insufflation material introduced into the body cavity is not retained in the body cavity;
an expansion material open channel in fluid communication with the internal surface of the internal buttress; and
an expansion material port in fluid communication with expansion material open channel.

2. The insufflation retention device of claim 1, further comprising:
a seam runs from an exterior surface of the passageway through to an interior surface of the passageway, wherein the seam extends all the way from the internal buttress to the external buttress.

3. The insufflation retention device of claim 1, wherein the internal buttress in an expanded state extends around an end of the insufflation retention device.

4. The insufflation retention device of claim 1, wherein the external buttress only has an expanded state and does not have an unexpanded state.

5. The insufflation retention device of claim 1, further comprising:
a shaft portion that extends into the internal buttress; and
a bevel that is a surface that is not substantially a right angle of a length of the shaft portion.

6. An insufflation retention device comprising:
an internal buttress configured to inhibit removal of the internal buttress from a body cavity through a body aperture of a body, wherein the internal buttress has an unexpanded state and an expanded state after introduction of an expansion material, and the internal buttress in an expanded state extends around an end of the insufflation retention device;
an external buttress coupled to the internal buttress, the external buttress configured to inhibit entry of the external buttress into the body cavity through the body aperture;
a passageway extending through the internal buttress and the external buttress that is configured for passage of a probe in contacting engagement with the body cavity, wherein the passageway is configured to be open without the probe present in the passageway such that an insufflation material introduced into the body cavity is not retained in the body cavity;
a seam runs from an exterior surface of the passageway through to an interior surface of the passageway, wherein the seam extends all the way from the internal buttress to the external buttress;
an expansion material open channel in fluid communication with the internal surface of the internal buttress; and
an expansion material port in fluid communication with expansion material open channel.

7. The insufflation retention device of claim 6, wherein the internal buttress in an expanded state extends around an end of the insufflation retention device.

8. The insufflation retention device of claim 6, wherein the external buttress only has an expanded state and does not have an unexpanded state.

9. The insufflation retention device of claim 6, further comprising:
a shaft portion that extends into the internal buttress; and
a bevel that is a surface that is not substantially a right angle of a length of the shaft portion.

10. The insufflation retention device of claim 6, further comprising:
a shaft portion that extends into the internal buttress; and
a knife edge channel with a rounded lead-in on the shaft portion, wherein the rounded lead-in is disposed on a first adjacent edge and a second adjacent edge, wherein the rounded lead-in is cut to form the seam.

11. The insufflation retention device of claim 10, wherein the knife edge channel is configured to closely match a contour of the internal buttress.

12. The insufflation retention device of claim 6, further comprising:
a probe side lead-in on the external buttress configured to nestle the probe.

13. The insufflation retention device of claim 12, wherein the probe side lean-in is characterized as a first probe side lead-in, further comprising:
a second probe side lead-in on the external buttress configured to nestle the probe.

14. The insufflation retention device of claim 6, further comprising:
an end lead-in on the external buttress configured to pivot the external buttress relative to the external buttress when the probe engages the external buttress.

15. The insufflation retention device of claim 6, further comprising:
introducer configured to decrease puncture force of the insufflation retention device insertion into the body cavity.

16. The insufflation retention device of claim 15, wherein the introducer has a leading edge configured to be rounded from an exterior surface to an interior surface of a shaft portion that extends into the internal buttress

17. A method of manufacturing an insufflation retention device comprising steps of:
manufacturing an internal buttress through a dip molding process;
manufacturing a handle; and
connecting the internal buttress to the handle.
